(19) United States
(12) Patent Application Publication
(10) Pub. No.: US 2005/0031615 A1
(43) Pub. Date: Feb. 10, 2005

(54) Method of inhibiting tumor growth with anti-tissue factor antibodies

(76) Inventors: G. Mark Anderson, Norristown, PA (US); Richard Tawadros, Norristown, PA (US); Cam Ngo, Philadelphia, PA (US); Marian Nakada, Malvern, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/855,664

(22) Filed: May 27, 2004

Related U.S. Application Data
(60) Provisional application No. 60/475,174, filed on May 30, 2003.

(51) Int. Cl. A61K 39/395

(52) U.S. Cl. 424/145.1

(57) ABSTRACT

A method of using tissue factor antagonists to treat proliferative diseases characterized by neovascularization such as cancer, rheumatoid arthritis, psoriasis, proliferative retinopathy, or macular degeneration. Tissue factor antagonists capable of rapid prevention of blood clotting via the extrinsic pathway are also capable of inhibiting tumor growth in mammals.

### CROSS REFERENCE TO RELATED APPLICATION

This application is a non-provisional application filed under 37 CFR 1.53(b)(1), claiming priority under 35 USC 119(e) to provisional application No. 60/475,174 filed May 30, 2003, the contents of which are incorporated herein by reference.

### BACKGROUND OF THE INVENTION

### Field of the Invention

The present invention relates to a method of using Tissue factor (TF) antagonists to treat cancer, by specifically preventing or inhibiting the growth of tumor cells. The invention more specifically relates to methods of treating such diseases by the use of TF antagonists such as antibodies directed toward TF, including specified portions or variants thereof, specific for at least one TF protein or fragment thereof, in an amount effective to inhibit the growth of tumors.

Tissue Factor (TF)

The coagulation of blood involves a cascading series of reactions leading to the formation of fibrin. The coagulation cascade consists of two overlapping pathways, both of which are required for hemostasis. The intrinsic pathway comprises protein factors present in circulating blood, while the extrinsic pathway requires tissue factor (TF), which is expressed on the cell surface of a variety of tissues in response to vascular injury (Davie et al., 1991, Biochemistry 30:10363). When exposed to blood, TF sets in motion a potentially explosive cascade of activation steps that result in the formation of an insoluble fibrin clot. TF has been investigated as a target for anticoagulant therapy.

TF is a single chain, 263 amino acid membrane glycoprotein that functions as a receptor for factor VII and VIIa and thereby initiates the extrinsic pathway of the coagulation cascade in response to vascular injury. TF is a transmembrane cell surface receptor which serves as the receptor as well as the cofactor for factor VIIa, forming a proteolytically active TF:VIIa complex on cell surfaces (Ruf et al, (1992) J.Biol. Chem 267:6375-6381). In addition to its role in maintaining hemostasis, excess TF has been implicated in pathogenic conditions. Specifically, the synthesis and cell surface expression of TF has been implicated in vascular disease (Wilcox et al., 1989, Proc. Natl. Acad. Sci, 86:2839) and gram-negative septic shock (Warr et al., 1990, Blood 75:1481).

TF Antagonists

Various anti-TF antibodies are known. For example, Carson et al, (1987, Blood 70:490-493) discloses a hybridoma producing monoclonal antibody prepared by immunizing mice with TF purified by affinity chromatography on immobilized factor VII. Ruf et al, (1991, Thrombosis and Haemostasis 66:529) characterized the anticoagulant potential of murine monoclonal antibodies against human TF. The ability of monoclonal antibodies that target the FVII binding site on TF, is dependent on their ability to compete with FVII for binding to TF and formation of the TF/VIIa complex, which is rapidly formed when TF contacts plasma. Such antibodies were thus relatively slow inhibitors of TF in plasma. One monoclonal antibody, TF8-5G9, was capable of inhibiting the TF/VIIa complex, thus providing an immediate anticoagulant effect in plasma. This antibody is disclosed in U.S. Pat. Nos. 6,001,978, 5,223,427, and 5,110,730. Ruf et al, suggested (supra) that mechanisms that inactivate the TF/VIIa complex, rather than prevent its formation, may provide strategies for interruption of coagulation in vivo. In contrast to other antibodies that inhibit factor VII binding to TF, TF8-5G9 shows only subtle and indirect effects on factor VII or factor VIIa binding to the receptor. TF8-5G9 binds to the extracellular domain of TF with a nanomolar binding constant to block the formation of the TF:F.VIIa:F.X ternary initiation complex (Huang et al, J. Mol. Biol. 275:873-894 1998).

Anti-TF monoclonal antibodies have been shown to inhibit TF activity in various species (Morissey et al., 1988, Thromb. Res. 52:247-260) and neutralizing anti-TF antibodies have been shown to prevent death in a baboon model of sepsis (Taylor et al, Circ. Shock, 33:127 (1991)), and attenuate endotoxin induced DIC in rabbits (Warr et al, (1990) Blood 75:1481).

WO 96/40921 discloses CDR-grafted anti-TF antibodies derived from the TF8-5G9 antibody. Other humanized or human anti-TF antibodies are disclosed in Presta et al, Thromb Haemost 85:379-389 (2001), EP1069185, WO 01/70984 and WO03/029295.

The Role of TF in Cancer

Tissue factor is also overexpressed on a variety of malignant tumors and isolated human tumor cell lines, suggesting a role in tumor growth and survival. TF is not produced by healthy endothelial cells lining normal blood vessels but is expressed on these cells in tumor vessels. It appears to play a role in both vasculogenesis, formation of new blood vessels, in the developing animal and angiogenesis, sprouting of new capillaries from existing arteries, in normal and malignant adult tissues. Inhibition or targeting of TF may therefore be a useful anti-tumor strategy that could affect the survival of TF overexpressing tumor cells directly by inhibiting TF mediated cellular signaling or other activities. In addition, this approach may prevent tumor growth indirectly via an antiangiogenic mechanism by inhibiting the growth or function of TF expressing intra-tumoral endothelial cells.

TF and Angiogenesis

Angiogenesis is the process of generating new capillary blood vessels, and results from activated proliferation of endothelial cells. Neovascularization is tightly regulated, and occurs only during embryonic development, tissue remodeling, wound healing and periodic cycle of corpus luteum development (Folkman and Cotran, Relation of vascular proliferation to tumor growth, Int. Rev. Exp. Pathol.′16, 207-248(1976)).

There is now considerable evidence that tumor growth and cancer progression requires angiogenesis and neovascularization, blood vessel growth and extension, in order to provide tumor tissue with nutrients and oxygen, to carry away waste products and to act as conduits for the metastasis of tumor cells to distant sites (Folkman, et al. N Engl J Med 285: 1181-1186, 1971 and Folkman, et al. N Engl J Med 333: 1757-1763, 1995). Nevertheless, tissue and tumor angiogenesis and neovascularization represent complex processes mediated by the interplay of cellularly produced factors: including TNFalpha, VEGF, and tissue factor. Studies show that the pathways leading to upregulation of VEGF and TF overlap (Chen J. et al. (2001) Thromb. Haemost. 86-334-5), two major players in the initiation of new blood vessel formation.

Endothelial cells normally proliferate much more slowly than other types of cells in the body. However, if the proliferation rate of these cells becomes unregulated, pathological angiogenesis can result. Pathological angiogenesis is involved in many diseases. For example, cardiovascular diseases such as angioma, angiofibroma, vascular deformity, atherosclerosis, synechia and edemic sclerosis; and opthalmological diseases such as neovascularization after cornea implantation, neovascular glaucoma, diabetic retinopathy, angiogenic corneal disease, macular degeneration, pterygium, retinal degeneration, retrolental fibroplasias, and granular conjunctivitis are related to angiogenesis. Chronic inflammatory diseases such as arthritis; dermatological diseases such as psoriasis, telangiectasis, pyogenic granuloma, seborrheic dermatitis, venous ulcers, acne, rosacea (acne rosacea or erythematosa), warts (verrucas), eczema, hemangiomas, lymphangiogenesis are also angiogenesis-dependent.

Vision can be impaired or lost because of various ocular diseases in which the vitreous humor is infiltrated by capillary blood. Diabetic retinopathy can take one of two forms, non-proliferative or proliferative. Proliferative retinopathy is characterized by abnormal new vessel formation (neovascularization), which grows on the vitreous surface or extends into the vitreous cavity. In advanced disease, neovascular membranes can occur, resulting in a traction retinal detachment. Vitreous hemorrhages may result from neovascularization. Visual symptoms vary. A sudden severe loss of vision can occur when there is intravitreal hemorrhage. Visual prognosis with proliferative retinopathy is more guarded if associated with severe retinal ischemia, extensive neovascularization, or extensive fibrous tissue formation. Macular degeneration, likewise takes two forms, dry and wet. In exudative macular degeneration (wet form), which is much less common, there is formation of a subretinal network of choroidal neovascularization often associated with intraretinal hemorrhage, subretinal fluid, pigment epithelial detachment, and hyperpigmentation. Eventually, this complex contracts and leaves a distinct elevated scar at the posterior pole. Both forms of age-related macular degeneration are often bilateral and are preceded by drusen in the macular region. Another cause of loss of vision related to angiogenic etiologies are damage to the iris. The two most common situations that result in the iris being pulled up into the angle are contraction of a membrane such as in neovascular glaucoma in patients with diabetes or central retinal vein occlusion or inflammatory precipitates associated with uveitis pulling the iris up into the angle (Ch. 99. The Merck Manual 17th Ed. 1999).

Rheumatoid arthritis, an inflammatory disease, also results in inappropriate angiogenesis. The growth of vascular endothelial cells in the synovial cavity is activated by the inflammatory cytokines, and results in cartilage destruction and replacement with pannus in the articulation (Koch A K, Polverini P J and Leibovich S J. Arthritis Rheum. 29, 471-479(1986); Stupack D G, Storgard C M and Cheresh D A, Braz. J. Med. Biol. Res., 32, 578-581(1999); Koch A K, Arthritis Rheum, 41, 951 962(1998)).

Psoriasis is caused by uncontrolled proliferation of skin cells. Fast growing cells require sufficient blood supply, and abnormal angiogenesis is induced in psoriasis (Folkman J., J. Invest. Derrnatol., 59, 40-48(1972)).

WO94/05328 discloses the use of anti-TF antibodies to inhibit the onset and progression of metastasis by abolishing the prolonged adherence of metastazing cells in the microvasculature thereby inhibiting metastasis, but does not disclose any effect on the growth of established tumor cells. Given the complexity in the factors regulating tumor vascularization as well as the incomplete understanding of the role of tissue factor as a receptor mediating cellular growth in both tumor growth and wound healing, it is possible that blockade of TF could play either a critical or a redundant role in the pathogenesis of cancer or other diseases characterized by inappropriate angiogenic activity.

Therefore, it would be of benefit to understand whether an antibody to TF could be used as primary or adjunctive therapy in the treatment of human cancers as well as other proliferative diseases accompanied by neovascularization and angiogenic mechanisms.

### SUMMARY OF THE INVENTION

The present invention relates to a method of using antagonists of TF, including antibodies directed toward TF, and specified portions or variants thereof specific for at least one TF protein or fragment thereof, to inhibit the growth of tumors in mammals. Such TF antagonists such as antibodies can act through their ability to bind to TF in a manner that prevents events associated with the growth of cancer tissue, particularly solid tumors.

In another aspect, the invention provides a method of treating a disease characterized by an increase in vascularized tissue, comprising administering a tissue factor antagonist in an amount effective to inhibit the increase of said tissue.

### BRIEF DESCRIPTION OF THE DRAWINGS

FIG. 1 is a graph showing tumor growth rates (volume change) of human breast cancer cells implanted subcutaneously into the flanks of nude mice and dosed with CNTO 859, irrelevant hIg, or HBSS once per week starting on Day 0.

FIG. 2 is a graph showing data from the same experiment as FIG. 1 for groups of mice dosed with CNTO 859 or hIg once per week starting at Day 14.

FIG. 3 is a graph showing the change in tumor volumes from either control animals, animals treated with either PBS or control human Ig and animals treated with CNTO 859.

FIG. 4 is a bar graph representing the mean and standard deviation of the final tumor volumes from either control animals, animals treated with either PBS or control IgG and animals treated with CNTO 859.

FIG. 5 shows the tumor incidence rate in animals treated with either PBS, control Ig or CNTO 859 beginning on the same day as the tumor cells were implanted.

FIG. 6 shows the tumor progression of MDA MB 231 xenografts as measured by volume in animals treated with either PBS, control human Ig or various dosages of CNTO 859. CNTO 859 was able to inhibit tumor growth at all concentrations. Tumor inhibition ranged from 90% at 0.1 mg/kg (p=0.0012 and p=0.0106, respectively, Wilcoxon two-sample test using t-distribution) to 95% at any concentration above that.

FIG. 7 is a scatter plot showing the distribution of final tumor volumes from animals treated with either PBS, control human Ig or various dosages of CNTO 859 (0.1, 1, 5, 10 and 20 mg/kg).

FIG. 8 is graph of tumor volumes over time for an experiment using human breast cancer cells MDA MB 231 xenografts implanted in mice orthotopically (in mammary tissue) and where the mice were treated with either PBS, control human Ig, CNTO 859 Ala/Ala or various dosages (0.01, 0.1 and 1 mg/kg) of CNTO 859 and CNTO 860.

FIG. 9 shows means and standard deviation of four of the groups from the same experiment shown in FIG. 8, showing only the controls and CNTO 859 and CNTO 860 at 0.1 mg/kg.

FIG. 10 is a graphical representation of each of the individual final tumor volumes and means from each group in the same experiment as FIG. 8.

FIG. 11 shows the tumor incidence data from the same experiment as in FIG. 8.

FIG. 12 is a graph showing tumor growth rates (volume change) of BxPC-3 human pancreatic tumor cells implanted in mice treated beginning the next day with CNTO 859. Tumor gowth is inhibited by 46.9% (p<0.0012).

FIG. 13 is a graph showing tumor growth rates (volume change) of BxPC-3 human pancreatic tumor cells implanted in mice when treating established tumors with CNTO 859. Tumor growth is inhibited by 35% (p<0.0001).

FIG. 14 is a bar graph showing PANC-1 human pancreatic tumor cells induced angiogenesis, as measured by vessel length in MATRIGEL in mice, is reduced by 88% (p<0.05) by a human anti-murine TF antibody (PHD 127).

### DETAILED DESCRIPTION OF THE INVENTION

The TF antagonists of the invention are useful in inhibiting and preventing tumor growth. A number of pathologies involving various forms of solid primary tumors are improved by treatment with TF antagonists in the method of the present invention.

Tumors

Both benign and malignant tumors, including various cancers such as, cervical, anal and oral cancers, stomach, colon, bladder, rectal, liver, pancreatic, lung, breast, cervix uteri, corpus uteri, ovary, prostate, testis, renal, brain/cns (e.g., gliomas), head and neck, eye or ocular, throat, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx, esophageal, larynx, kidney and lymphoma, among others may be treated using anti-TF antibodies of the present invention.

Thus, the present invention provides a method for modulating or treating at least one malignant disease in a cell, tissue, organ, animal or patient, including, but not limited to, at least one of: breast carcinoma, colorectal carcinoma, renal cell carcinoma, pancreatic carcinoma, prostatic carcinoma, nasopharyngeal carcinoma, malignant histiocytosis, paraneoplastic syndrome/hypercalcemia of malignancy, solid tumors, adenocarcinomas, sarcomas, malignant melanoma, hemangioma, metastatic disease, and the like. Such a method can optionally be used in combination with, by administering before, concurrently or after administration of such TF antagonist, radiation therapy, an anti-angiogenic agent, a chemotherapeutic agent, a farnesyl transferase inhibitor, a protesome inhibitor or the like.

TF Antagonists

As used herein, the term “TF antagonists” refers to a substance which inhibits or neutralizes the activity of TF. Such antagonists accomplish this effect in a variety of ways. One class of TF antagonists will bind to TF protein with sufficient affinity and specificity to neutralize the effect of TF. Included in this class of molecules are antibodies and antibody fragments (such as for example, F(ab) or F(ab′)₂ molecules). Thus, as used herein “antibody” refers to immunoglobulin molecules and immunologically active portions of immunoglobulin molecules. Another class of TF antagonists are fragments of TF protein, muteins or small organic molecules i.e. peptidomimetics, that will bind to TF ligands, thereby inhibiting the activity of TF or its ability to cause intracellular signalling. The TF antagonist may be of any of these classes as long as it is a substance that inhibits TF anti-tumor activity or anti-angiogenic activity. TF antagonists include TF antibody, modified TF, antisense TF and partial peptides of TF.

In a particularly preferred embodiment, the TF antagonist is a murine, chimeric, humanized or human monoclonal antibody or fragment thereof which has one of the following properties: prevents factor VIIa binding to TF thereby preventing coagulation to proceed, prevents the formation of TF:FVII:FX complex, or prevents TF signaling via its intracellular domain. Such antibodies are known in the art and may be employed in the method of the present invention. Murine monocolonal antibodies to TF are known as in, for example U.S. Pat. Nos. 6,001,978, 5,223,427, and 5,110,730. WO 96/40921 discloses CDR-grafted anti-TF antibodies derived from the TF8-5G9 antibody in which the complementary determining regions (CDR's) from the variable region of the mouse antibody TF8-5G9 are transplanted into the variable region of a human antibody and joined to the constant region of a human antibody. Other humanized anti-TF antibodies capable of preventing TF anti-coagulant and receptor mediated activies are disclosed in Presta et al, Thromb Haemost 85:379-389 (2001) and EP1069185. Each of the foregoing references are incorporated by reference into the present application.

Compositions and Their Uses

The neutralizing anti-TF monoclonal antibody can be used to inhibit tumor growth in accordance with the invention. The individual to be treated may be any mammal and is preferably a human patient in need of such treatment. The amount of monoclonal antibody administered will vary according to the purpose it is being used for and the method of administration.

The TF antibodies of the present invention may be administered by any number of methods that result in an effect in tumor tissue in which growth is desired to be prevented or halted. Further, the anti TF antibodies of the invention need not be present locally to impart an antitumor effect, therefore, they may be administered wherever access to body compartments or fluids containing TF is achieved. In the case of malignant tissues, these methods may include direct application of a formulation containing the antibodies. Such methods include intravenous administration of a liquid composition, subcutaneous or transdermal administration of a liquid or solid formulation, oral, topical administration, or interstitial or inter-operative administration.

Administration may also be oral or by local injection into a tumor or tissue but generally, the monoclonal antibody is administered intravenously. Generally, the dosage range is from about 0.01 mg/kg to about 12.0 mg/kg. This may be as a bolus or as a slow or continuous infusion which may be controlled by a microprocessor controlled and programmable pump device.

Alternatively, DNA encoding preferably a fragment of said monoclonal antibody may be isolated from hybridoma cells and administered to a mammal. The DNA may be administered in naked form or inserted into a recombinant vector, e.g., vaccinia virus in a manner which results in expression of the DNA in the cells of the patient and delivery of the antibody.

The monoclonal antibody used in the method of the present invention may be formulated by any of the established methods of formulating pharmaceutical compositions, e.g. as described in Remington's Pharmaceutical Sciences, 1985. For ease of administration, the monoclonal antibody will typically be combined with a pharmaceutically acceptable carrier. Such carriers include water, physiological saline, or oils.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Except insofar as any conventional medium is incompatible with the active ingredient and its intended use, its use in any compositions is contemplated.

The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use.

Combinations with TF Antagonists

The method may be carried out by combining the TF antagonists of the invention with one or more other agents having anti-tumor effect or a dissimilar mechanism of inhibiting in vivo angiogenesis, including, but not limited to chemotherapeutic agents.

Further, the TF antibody can be combined with one or more anti-angiogenic agents. Angiogenesis is characterized by the invasion, migration and proliferation of smooth muscle and endothelial cells. The αvβ3 integrin (also known as the vitronectin receptor) is known to play a role in various conditions or disease states including tumor metastasis, solid tumor growth (neoplasia), osteoporosis, Paget's disease, humoral hypercalcemia of malignancy, angiogenesis, including tumor angiogenesis, retinopathy, including macular degeneration, arthritis, including rheumatoid arthritis, periodontal disease, psoriasis and smooth muscle cell migration (e.g. restenosis).

The adhesion receptor integrin αvβ3 binds vitronectin, fibrinogen, von Willebrand Factor, laminin, thrombospondin, and other like ligands. It was identified as a marker of angiogenic blood vessels in chick and man and plays a critical role in angiogenesis or neovascularization. Antagonists of αvβ3 inhibit this process by selectively promoting apoptosis of cells in neovasculature. Therefore, αvβ3 antagonists would be useful therapeutic targets for treating such conditions associated with neovascularization (Brooks et al., Science, Vol. 264, (1994), 569-571). Additionally, tumor cell invasion occurs by a three step process: 1) tumor cell attachment to extracellular matrix; 2) proteolytic dissolution of the matrix; and 3) movement of the cells through the dissolved barrier. This process can occur repeatedly and can result in metastases at sites distant from the original tumor. The αvβ3 integrin has been shown to play a role in tumor cell invasion as well as angiogenesis.

As the antagonists of αvβ3 and neutralizing anti-TF antibodies both target tumors but act through different mechanisms, the combination of anti-integrin antibodies with anti-TF antibodies should result in a particularly potent and effective combination therapy with little normal tissue toxicity. Thus, in one embodiment of the present invention, there is provided a method of inhibiting the growth of tumors which comprises administering a combination of an integrin antagonist and an anti-TF antibody in a patient in need of such treatment. Other antibodies which selectively bind integrins or integrin subunits, especially those that bind the alphaV subunit, are disclosed in U.S. Pat. Nos. 5,985,278 and 6,160,099. Mabs that inhibit binding of alphaVbeta3 to its natural ligands containing the tripeptide argininyl-glycyl-aspartate (RGD) are disclosed in U.S. Pat. No. 5,766,591 and WO0078815. Other antibodies that prevent alphaV-subunit containing integrins from binding to vitronection, fibronectin, or other ligands have similar utility in preventing angiogenesis. Such antibodies include the antibody known at GEN 095 or CNTO 95 and described in applicants co-pending application published as WO02012501.

In accordance with the invention, other known anti-angiogenesis agents such as thalidomide may also be employed in combination with an anti-TF antibody.

Abbreviations

ATCC—American Type Culture Collection
CO₂—carbon dioxide
DMEM—Dulbeccos Modified Eagles Medium
EDTA—ethylenediamine tetra-acetic acid
FBS—fetal bovine serum
FVIIa—Factor VIIa (activated FVII)
FX—Factor X (inactive)
FXa—Factor Xa (activated FX)
hIg—Human Ig
LNN—2 mM L-glutamine, 1 mM sodium pyruvate, 0.1 mM non-essential AAs
PBS—phosphate buffered saline
SQ—subcutaneous
IV—intravenously
IP—intraperitoneal
TF—Tissue Factor
   While having described the invention in general terms, the embodiments of the invention will be further disclosed in the following examples.

### EXAMPLE 1

### Inhibition of Tumor Growth in Breast Carcinoma Xenografts

This example demonstrates the ability of anti-tissue factor IgG antibody to inhibit tumor growth of MDA-MB-231 breast carcinoma xenografts implanted in nude mice.

Materials and Methods (50) 4-6 week-old female Nude mice (Crl: NU/NU-CD1) from Charles River Laboratories were obtained and acclimated for 10-14 days prior to experimentation. Mice were maintained in the animal facility at Centocor, Inc. in accordance to the National Institutes of Health Guide for the Care and Use of Laboratory Animals.

The human breast carcinoma cell line MDA-MB-231 was obtained from ATCC (Rockville, Md., Catalog #HTB-26). Cells were cultured in DMEM media supplemented with 25 mM HEPES, 10% FBS and 1% LNN at 37° C., 5% CO₂. Cells were harvested at log phase growth with trypsin-EDTA and resuspended in sterile HBSS at 5×10⁷ cells/mL.

Antibodies: CNTO 859, CDR grafted TF8-5G9 antibody disclosed in WO96/40921, stock concentration 3.75 mg/mL; hIg, ZLB Bioplasma, AG, Beme Switzerland. Stock concentration 30 mg/niL in sterile USP water). All test articles were set at a working concentration of 2 mg/mL in sterile HBSS. All test articles and controls will have LAL values <1.0 EU/mg.

On day 0, mice were randomly assigned to each of 5 groups, 10 mice per group. Cells were implanted subcutaneously into the flank of nude mice at a concentration of 5×10⁶ MDA-MB-231 tumor cells in a volume of 0.2 mL (0.1 mL cells in HBSS mixed with 0.1 mL of Matrigel®). The groups of mice were dosed once per week as proscribed in the TABLE 1.

TABLE 1

| | | | Initial | Antibody | |
|---|---|---|---|---|---|
| Group No. | | Start | Dose | Concen- | Maintenance |
| (Treatment) | N | Dosing | (mg/kg) | tration | Dose (mg/kg) |
| 1. HBSS | 10 | Day 0 | 0.2 mL | N/A | 0.1 mL |
| 2. hIg Control | 10 | Day 0 | 20 | 2 mg/mL | 10 mg/kg |
| 3. CNTO 859 IgG | 10 | Day 0 | 20 | 2 mg/mL | 10 mg/kg |
| 4. hIg Control | 10 | Day 14 | 20 | 2 mg/mL | 10 mg/kg |
| | | (or avg. | | | |
| | | tumor | | | |
| | | size of | | | |
| | | 100 mm³) | | | |
| 5. CNTO 859 IgG | 10 | Day 14 | 20 | 2 mg/mL | 10 mg/kg |
| | | (or avg. | | | |
| | | tumorsize | | | |
| | | of | | | |
| | | 100 mm³) | | | |

On Day 0 of the study, 50 study mice are placed into 5 groups (10 mice/group, according to Table 1). All animals are injected with 0.2 mL MDA-MB-231 cell suspension containing 5×10⁶ cells (1:1 mixture of cell suspension in HBSS with Matrigel®) subcutaneously on the right side of the rib cage area. On Day 0, all animals in Groups 1, 2, and 3 received an intraperitoneal injection of 10 mL/kg of test article or HBSS (Table 1). Animals in Groups 4 and 5 received an intraperitoneal injection of 10 mL/kg of test article on Day 14 or at an average tumor size of 100 mm³). After the initial intraperitoneal injection all animals receive weekly intraperitoneal (5 mL/kg) doses of test article or control—until day 80.

The dose of 20 mg/kg for the first dose and 10 mg/kg for subsequent doses was calculated based on the most recent previous body weight value. Animals were dosed IP on Monday every week until day 80 or until the tumors reach a volume of 2,000 mm³.

Animal weights and tumor volumes were measured once weekly until the end of the study. Animals were weighed beginning on day 0 whereas tumor volumes were recorded only once palpable. Tumors were measured in three dimensions using calipers and tumor volumes were calculated based on the formula V=(L×W×T)/2, where L=length, W=width and T=thickness.

Study termination was planned when tumors reached a mean volume of ˜2000 mm³, with an option to extend the study on the condition that animal welfare was not being compromised. At termination, animals were euthanized via CO₂ asphyxiation and tumors were excised and weighed. Individual tumors were then bisected with one half snap frozen in OCT and the other half fixed in 10% formalin. Serum samples were taken from each animal at termination via cardiac puncture.

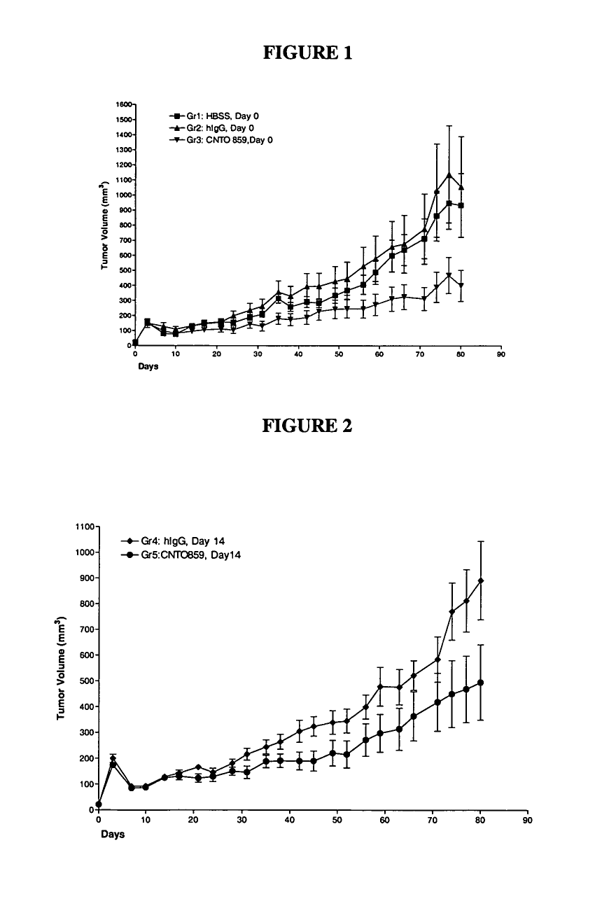

Results The growth rate of each treatment group was plotted as a function of tumor volume (mm³) versus time (days after implantation) (FIG. 1 and 2). There was a 100% take rate in all groups. Treatment of animals at day 0 (FIG. 1) or day 14 (FIG. 2) with hIg control did not significantly impact tumor growth relative to the HBSS negative control (P=0.779, P=0.979, respectively).

Tumor growth in animals treated with CNTO 859 at day 0 was inhibited by 62% (P<0.0001) (FIG. 1). When treatment with CNTO 859 was initiated on day 14, when the average tumor size was 100 mm , tumor growth rates were inhibited by 47% (P<0.0001) (FIG. 2).

The overall combined treatment effect of CNTO 859 was also significant relative to the overall treatment effect of hIg control (P<0.0001). Day to day significance, defined as P<0.005, was generally achieved after day 17 of treatment.

The results demonstrate that CNTO 859 inhibited tumor growth rates up to 62%. This is the first time it has been demonstrated that an anti-human Tissue Factor IgG antibody has the ability to inhibit human derived tumor growth in vivo. Both early (day 0) and late (day 14) treatment with CNTO 859 significantly inhibited tumor growth rates.

### EXAMPLE 2

### Effect of Anti-tf Antibody on Human Breast Carcinoma in an Orthotopic Xenograft Model

In this example, an orthotopic tumor growth model using the human breast carcinoma cell line, MDA MB 231, injected into the mammary fat pad of SCID/Beige mice was used to test the anti-tumor effect of CNTO 859. In addition, the effect of variations on the structure of anti-tissue factor antibody were compared: one differing in human class identity CNTO 859 (IgG4) and CNTO 860 (IgG1); and modification of the FcR binding region CNTO 859 designated CNTO 859 ala/ala.

Materials and Methods Four week-old female SCID/Beige mice (C.B.-17/IcrCrl-scid-bgBR) from Charles River Laboratories were obtained and acclimated for 10-14 days prior to experimentation. Mice were housed 7-8/cage in filter top cages and supplied with autoclaved food and acidified water containing Bactrum (0.13 mg/mL trimethoprim/0.66 mg/mL sulfamethoxazole) ad libitum. Animals were identified by individually numbered ear tags placed 5 days prior to the start of the study. Cage cards labeled with source, sex, number of animals, animal ID numbers, group number, treatments, study number and IACUC protocol number were affixed to the cages. All animal studies were carried out in the vivarium at Centocor, Inc., Radnor, Pa. in accordance to the National Institutes of Health Guide for the Care and Use of Laboratory Animals.

The human breast carcinoma cell line MDA MB 231 was obtained from the cell repository at Centocor and have been deemed sterile and mycoplasma-free. Cells were cultured in DMEM media supplemented with 10% FBS and 1% LNN at 37° C., 5% CO₂. Cells were harvested at log phase growth with trypsin-EDTA and resuspended at 5×10⁷ cells/mL in serum-free DMEM and were implanted into the (Rt inguinal #2/3) mammary fat pad in a volume of 50 uLs.

Test and Control Antibodies were as follows: CNTO 859, 3.75 mg/mL stock concentration; CNTO 859, 10.29 mg/mL stock; CNTO 860, 2.4 mg/mL stock; CNTO 859 Ala/Ala, C1081, lmg/mL stock; Human Ig, ZLB Bioplasma AG, Berne Switzerland, 30 mg/mL stock concentration.

Antibodies were supplied at appropriate concentrations in PBS. All control and test articles have been endotoxin tested to be <1EU/mg and will be administered intravenously.

Animals were randomized 7-8 mice/group. On day 0, 2.5×10⁶ MDA MB 231 cells were injected into the mammary fat pad of the animals in a volume of 50 uLs using a 30 g needle. Intravenous antibody therapy commenced on day 3. Dosing regimens and concentrations for each of the three studies are detailed in Tables 2, 3 and 4, respectively.

TABLE 2

| Table 1. | | | | |
|---|---|---|---|---|
| Group | Mice/Group | Cells | Antibody | Weekly × 8 |
| 1 | 8 | — | PBS | 200 uLs |
| 2 | 8 | 231 | PBS | 200 uLs |
| 3 | 8 | 231 | CNTO 859 | 20 mg/kg |
| 4 | 8 | 231 | Human Ig | 20 mg/kg |

TABLE 3

| Table 2. | Mice/ | | | |
|---|---|---|---|---|
| Group | Group | Cells | Antibody | Weekly × 8 |
| 1 | 8 | 231 | PBS | 200 uLs |
| 2 | 8 | 231 | Human Ig | 20 mg/kg |
| 3 | 8 | 231 | CNTO 859 | 0.1 mg/kg |
| 4 | 8 | 231 | CNTO 859 | 1 mg/kg |
| 5 | 8 | 231 | CNTO 859 | 5 mg/kg |
| 6 | 8 | 231 | CNTO 859 | 10 mg/kg |
| 7 | 8 | 231 | CNTO 859 | 20 mg/kg |

TABLE 4

| Table 3. | Mice/ | | | |
|---|---|---|---|---|
| Group | Group | Cells | Antibody | Weekly × 8 |
| 1 | 7 | 231 | PBS | 200 uLs |
| 2 | 7 | 231 | Human Ig | 1 mg/kg |
| 3 | 7 | 231 | CNTO 859 | 1 mg/kg |
| 4 | 7 | 231 | CNTO 859 | 0.1 mg/kg |
| 5 | 7 | 231 | CNTO 859 | 0.01 mg/kg |
| 6 | 7 | 231 | CNTO 860 | 1 mg/kg |
| 7 | 7 | 231 | CNTO 860 | 0.1 mg/kg |
| 8 | 7 | 231 | CNTO 860 | 0.01 mg/kg |
| 9 | 7 | 231 | CNTO 859 | 1 mg/kg |
| | | | Ala/Ala | |

The mice were weighed and tumor volumes were recorded once weekly for a period of 8-9 weeks. Tumor volumes were calculated as (L×W²)/2. The study terminated approximately eight to nine weeks after tumor cell inoculation. In the case that any animal experiences rapid weight loss, respiratory difficulty or becomes moribund prior to the termination point, that animal was euthanized by the Study Coordinator. Animals were euthanized via CO₂ asphyxiation and then weighed. Lungs and axillary lymph nodes were surgically removed, rinsed in cold PBS, blotted, weighed and immediately fixed in Bouin's solution. Primary tumors were resected, weighed and then fixed in BZT solution for histological analysis.

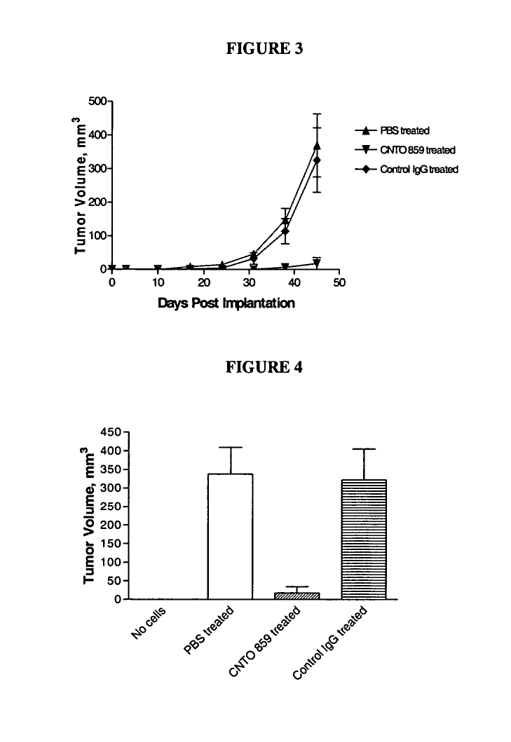

Primary Anti-Tumor Effect Tumor volumes were monitored and recorded once weekly during the study. At termination, primary tumors were surgically resected from CO₂ euthanized SCID/Beige mice and weighed. Tumor volumes and final mass were plotted over time (FIG. 3 and 4). Tumor growth was inhibited by over 95% when animals were treated with CNTO 859 relative to either the PBS or control IgG treated animals. (p=0.0039 and p=0.0126, two-tailed parametric t test, n=8).

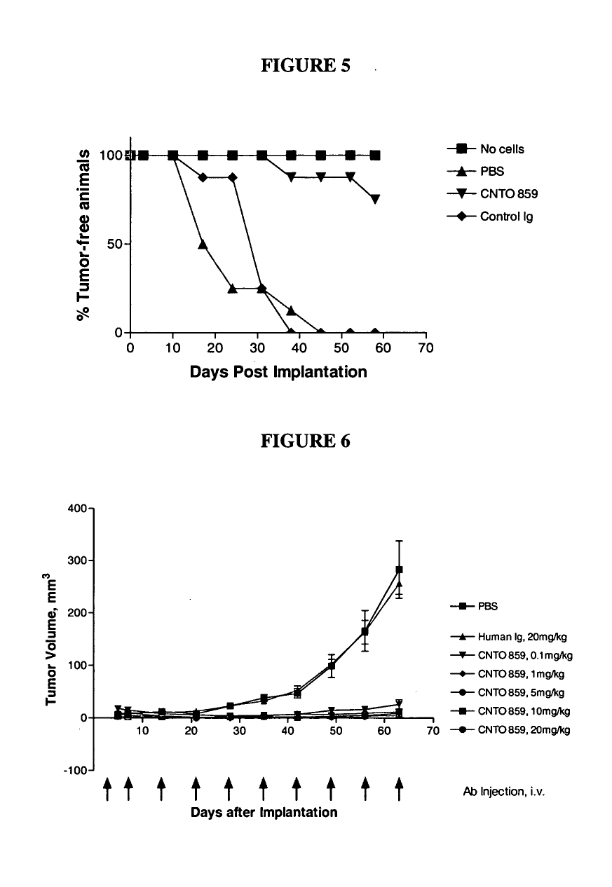

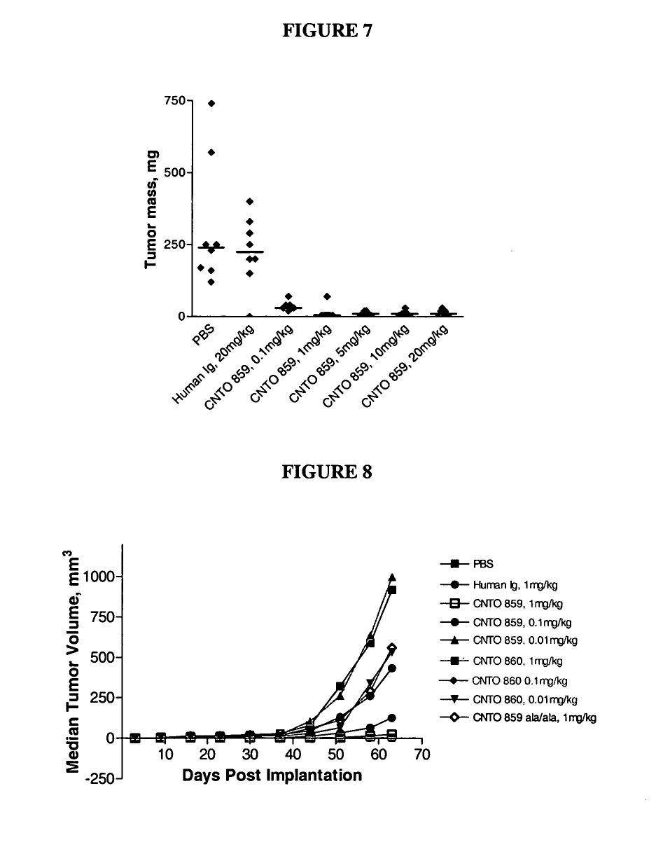

In a second study, the effect of dose was examined. CNTO 859 inhibited tumor growth at doses as low as 0.1 mg/kg, given once weekly. The was a significant reduction in tumor progression as tumor volume change (FIG. 6) and individual final tumor weights (FIG. 7) in animals treated with either 0.1, 1, 5, 10 or 20 mg/kg of CNTO 859 compared to PBS and Human Ig control groups.

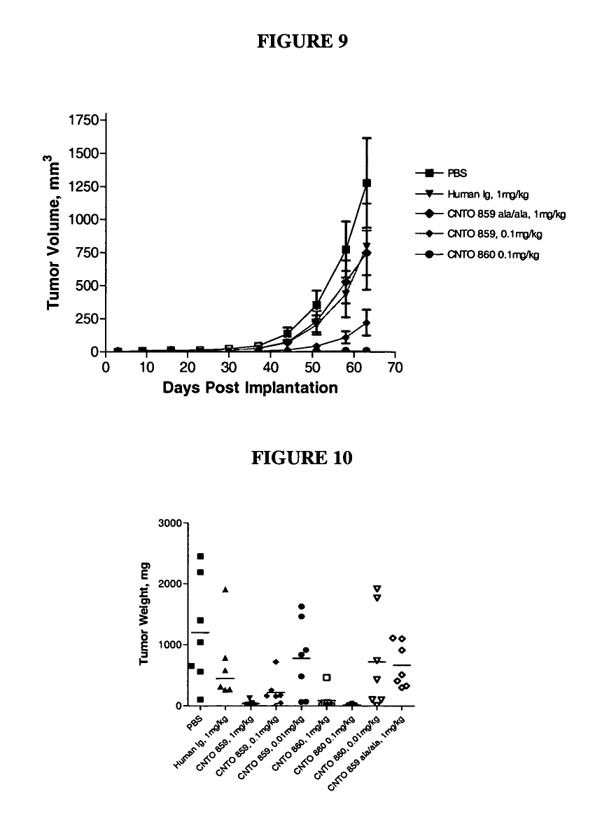

In a comparison study between CNTO 859 and CNTO 860 at three concentrations, it was shown that the IgG1 version of our anti-tissue factor antibody was superior in preventing not only tumor growth, but also tumor incidence (FIGS. 8-11). Tumor volumes from each of the respective groups are shown in (FIG. 8) as mean tumor weight in the group over time (FIG. 8) and as individual final weights and mean (FIG. 10) and in.

Effect on Tumor Incidence. CNTO 859 therapy also showed a marked difference in tumor incidence in treated animals. In the first study, cells were able to adhere and seed in the mammary fat pad as observed by the palpation of a nodule at the injection site, but were too small to measure until approximately day 38, when one tumor was of measurable size. In contrast, measurable tumors appeared in PBS or control human Ig treated animals beginning on day 17. FIG. 5 shows the tumor incidence rate in animals treated with either PBS, control Ig or CNTO 859. Results from this orthotopic MDA MB 231 tumor growth model indicate that CNTO 859 is a highly effective inhibitor of tumor incidence, growth and progression. Compared with a vehicle control or Ig control, CNTO 859 reduced tumor growth by 95% (p=0.0039 and p=0.0126, two-tailed parametric t test, n=8) and tumor incidence by 87.5% (p=0.0017 vs PBS and p=0.0086 vs control human Ig, two-tailed parametric t test, n=8).

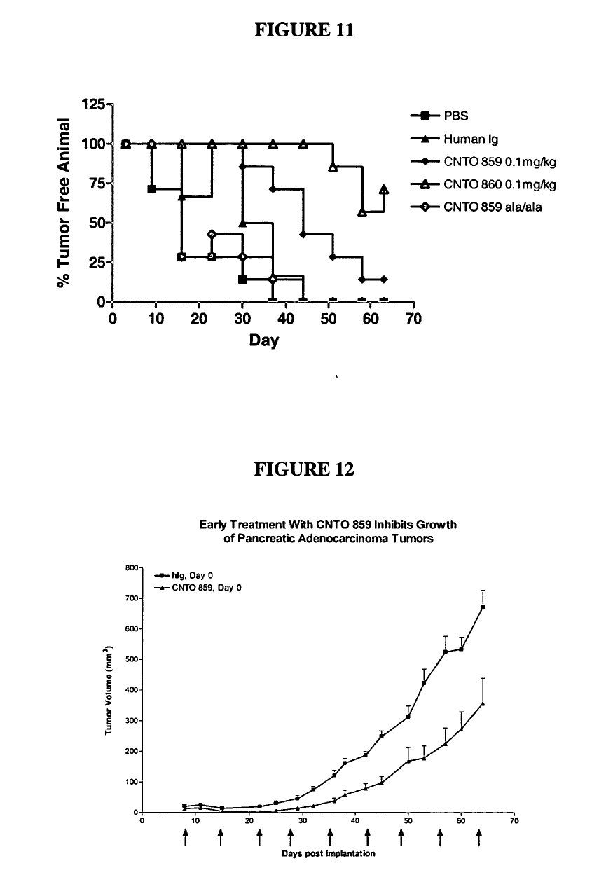

In the comparison study between CNTO 859 and CNTO 860, it was evident when using a dosage of 0.1 mg/kg, CNTO 860 was able to delay initial tumor onset by 44 and 37 days compared to the PBS and Human Ig control groups. Similarly, CNTO 859 was able to delay initial tumor onset by 23 and 16 days. Furthermore, by the end of the study, over 70% of the animals were tumor-free in the CNTO 860 group compared to only 15% in the CNTO 859 group. All animals in the PBS, Human Ig and CNTO 859 ala/ala groups had tumors by day 44 (FIG. 11).

Summary CNTO 859 and two variants were compared for efficacy in preventing tumor growth and progression in a series of experiments using this xenograft model. In the first study, CNTO 859 was highly efficacious in preventing tumor growth when given once weekly starting on day 3 post-tumor implantation at a concentration of 20 mg/kg, resulting in a 95% growth inhibition rate compared to either the PBS or control Ig treatment groups (p=0.0039 and p=0.0126, respectively). We also observed an 87.5% reduction in tumor incidence in animal treated with CNTO 859 compared to either the PBS or control Ig treatment groups (p=0.0017 and p=0.0086, respectively).

In a second study, CNTO 859 was administered at a series of dosages ranging from 0.1 mg/kg to 20 mg/kg once weekly. Results show that anti-TF monoclonal antibody therapy with CNTO 859 was highly efficacious in slowing tumor progression, even at a very low dose of 0.1 mg/kg, resulting in over 90% tumor inhibition compared to either the PBS or control Ig treatment groups (p=0.0012 and p=0.0106, respectively, Wilcoxon two-sample test using t-distribution). Dosages of 1, 5, 10 and 20 mg/kg significantly inhibited tumor growth by over 95%.

Lastly, in a separate study, the efficacy of CNTO 859 was evaluated against CNTO 860, an IgG1 version of CNTO 859, and the ADCC minimized version, CNTO 859 ala/ala. Doses of 0.01 mg/kg of either the IgG4 or IgG1 therapeutic antibody was no different than PBS, Human Ig control or CNTO 859 Ala/Ala. In contrast, a dose of 1 mg/kg of either CNTO 859 or CNTO 860 was able to inhibit tumor growth by over 95%. Interestingly, at the 0.1 mg/kg dose level, the effect CNTO 859 versus CNTO 860 is distinguished as CNTO 860 inhibited tumor growth by over 95% even at this low dose while CNTO 859 treated tumors were showing signs of escape from therapy, resulting in only ˜85% inhibition. In addition, CNTO 860 was more effective than CNTO 859 at slowing tumor progression when used at 0.1 mg/kg, presumably due to additional ADCC activity.

### EXAMPLE 3

### Inhibition of Tumor Growth in Pancreatic Adenocarcinoma Xenografts

In this example we demonstrate the effect of an anti-tissue factor antibody on growth inhibition of the pancreatic adenocarcinoma cell line, BxPC-3 grown in the flank of SCID mice. CNTO 859 was dosed once weekly at 10 mg/kg following an initial loading dose of 20 mg/kg. In groups where therapy with CNTO 859 was initiated 1 day, post tumor implantation, growth of BxPC-3 tumors was inhibited by 46.9% (p<0.001). In groups where treatment was initiated at mean tumor volume of 50-100 mm³, tumors were mildly inhibited, but statistical significance was not realized (p=0.6280).

Materials and Methods Six to eight week-old female SCID mice from Charles River Laboratories (Wilmington) were obtained and acclimated for 10-14 days prior to experimentation. Mice were housed 10/cage in filter top cages, and supplied with autoclaved food and acidified water, containing Bactrum (0.13 mg/mL trimethoprim/0.66 mg/mL sulfamethoxazole) ad libitum. Individually numbered ear tags placed 7 days prior to the start of the study identified animals. Cage cards labeled with source, sex, number of animals, animal ID numbers, group number, treatments, study number and IACUC protocol number were affixed to the cages. All animal studies were carried out in the vivarium at Centocor, Inc., Radnor, Pa. in accordance to the National Institutes of Health Guide for the Care and Use of Laboratory Animals.

The human BxPC-3 pancreatic adenocarcinoma cell line was obtained from ATCC (Rockville, Md., Catalog #CRL-1687). They were tested to be free of viral or mycoplasma contamination, and banked by Centocor's Cell Biology Services. Cells were cultured in RPMI 1640 media supplemented with 10% FBS and 1% LNN at 37° C., 5% CO₂. Cells were harvested at log phase growth with trypsin-EDTA and resuspended in sterile PBS at 1.5×10⁷ cells/mL.

CNTO 859 produced at Centocor, Inc., was used at a stock concentration 3.75 mg/mL; hIg, ZLB Bioplasma, AG, Berne Switzerland was used at stock concentration 30 mg/mL in sterile USP water), and PBS, pH7.1, Gibco BRL. All test articles were diluted to a working concentration of 2 mg/mL in sterile HBSS.

On day 0, mice were randomly assigned to each of 5 groups, 10 mice per group. Mice were inoculated subcutaneously with 3×10⁶ BXPC-3 tumor cells in the flank in a volume of 0.2 mL PBS or PBS alone (Group 1). The therapeutic regimen is detailed in Table 5.

On day 1, animals in Group 1 received 0.2 mL PBS (i.p.), Group 2 received 20 mg/kg hIg control antibody, and Group 3 received 20 mg/kg CNTO 859 antibody. Antibodies were subsequently dosed once every 7 days at 10 mg/kg. PBS was given once every seven days at 0.2 mL. Animals in Group 4 received treatment with 20 mg/kg CNTO 859 dosed i.p. once tumors reached aproximately 50 mm³ to 100 mm3 and then once every 7 days thereafter at 10 mg/kg. Animals in Group 5 received treatment with 20 mg/kg human Ig dosed i.p. when tumors reached approximately 50 mm3 to 100 mm³ in size and then once every 7 days thereafter at 10 mg/kg.

TABLE 5

| | | | | Sub- | |
|---|---|---|---|---|---|
| | | | Initial | sequent | |
| | | Antibody | Antibody | Antibody | |
| | | Conc. | Dose | Dose | Initiation |
| Group | Treatment | (mg/mL) | (mg/kg) | (mg/kg) | Criteria |
| 1 | PBS | 0 | 0 | 0 | Day 1 |
| 2 | hIg | 2 | 0 | 10 | Day 1 |
| 3 | CNTO 859 | 2 | 0 | 10 | Day 1 |
| 4 | CNTO 859 | 2 | 0 | 10 | 50-100 mm³ |
| 5 | hIg | 2 | 0 | 10 | 50-100 mm³ |

Animal weights and tumor volumes were monitored once weekly until the end of the study. Animals were weighed beginning on day 0 whereas tumor volumes were only recorded once palpable. Tumors were measured in three dimensions using calipers and tumor volumes were calculated based on the formula V=(L×W×T)/2, where L=length, W=width and T=thickness.

Study termination was planned when tumors reached a mean volume of ˜2000 mm³, with an option to extend the study on the condition that animal welfare was not being compromised. At termination, animals were euthanized via CO₂ asphyxiation and tumors were excised and weighed. Individual tumors were then bisected, with one half snap frozen in OCT and the other half fixed in 10% formalin. Serum samples were taken from each animal at termination via cardiac puncture.

Results The tumor growth rate of each treatment group, plotted as tumor volume (mm³) versus time (days after implantation) is shown (FIG. 12). An average tumor volume of 40 mm³ was reached on day 29 for mice treated with hIg on day 1. However, in mice treated with CNTO 859 the average tumor volume did not reach ˜40 mm³ until day 36.

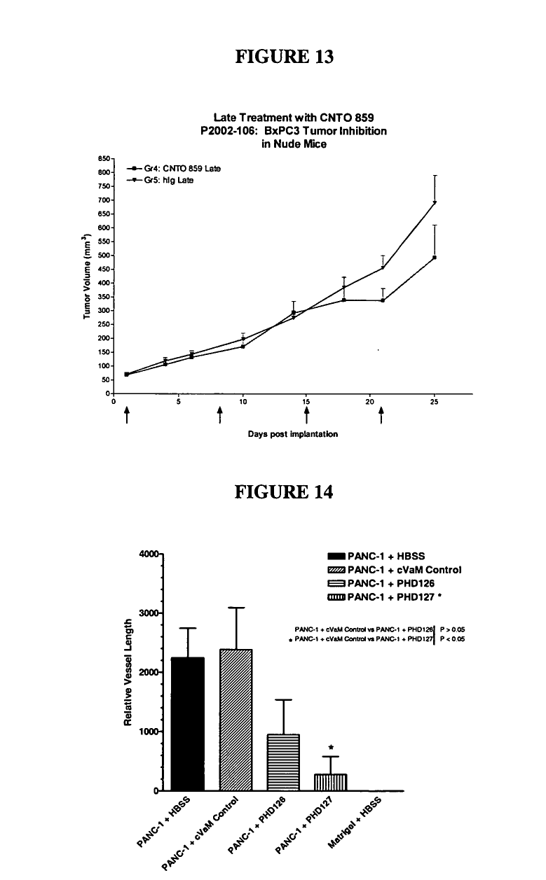

Treatment of animals at day 1 with CNTO 859 resulted in a 46.9% inhibition of tumor growth (P<0.0001) (see FIG. 12). Treatment of tumors at approximately 50-100 mm³ resulted in a mild but not significant inhibition of 28% (P=0.6280) (see FIG. 13).

It should be noted that in the 14 day CNTO 859-treated group, two of the seven mice had to be sacrificed at 18 days after tumor implantation, due to ulcerating tumors. The final tumor volume of these animals was 129.97 mm³ and 461.10 mm³, respectively. In the 14-day hIg treatment group, eight animals were reduced to seven on day 18. For these reasons, the late treatment arm of the study was terminated on day 25.

Summary CNTO 859 inhibited tumor growth rates up to 46.9%. To the best of our knowledge, this is the first time it has been demonstrated that an anti-human Tissue Factor antibody has the ability to inhibit tumor growth of a pancreatic adenocarcinoma. Early treatment with CNTO 859 resulted in significant inhibition of tumor growth rates (P<0.0001) relative to hIg control antibody. Late treatment with CNTO 859 inhibited tumor growth rates by 28%, but was not statistically significant (P=0.6280).

### EXAMPLE 4

### Inhibition of Angiogenesis Induced by Panc-1 Pancreatic Adenocarcinoma in a Matrigel Angiogenesis Model

In this example we demonstrate the effect of anti-murine Tissue Factor antibody in inhibiting angiogenesis induced by PANC-1 human pancreatic adenocarcinoma cells in a Matrigel angiogenesis model. Antibodies against murine Tissue Factor were obtained using directed selection of a phage library of human antibody sequences which were converted to full length mIgG2a antibodies. The human anti-mouse TF antibodies, designated PHD 126 and PHD 127, both inhibit activity of mouse Tissue Factor by a mechanism identical to that of CNTO 859 and its analogs. Specifically, PHD 126 and PHD 127 are competitive inhibitors of FX, and inhibit formation of a ternary complex formed by TF, factor VIIa and the enzymatically active factor Xa. Both antibodies inhibit coagulation promoted by murine TF and are selective for murine TF over human TF.

Materials and Methods Four- to six-week-old female Nude (Nu/Nu CD1) mice from Charles River Laboratories (Wilmington) were obtained and acclimated for 10-14 days prior to experimentation. Mice were group housed (7/cage) in filter top plastic cages and supplied with autoclaved food and water. A numbered ear tag or tattoo, placed at least 7 days prior to the start of the study, individually identified animals. All animal cages were identified with the IACUC protocol number, study number, animal numbers and treatment. All animal studies were carried out in the vivarium at Centocor, Inc., Radnor, Pa.

The human pancreatic adenocarcinoma cell line PANC-1 was obtained from ATCC (American Type Culture Collection, Rockville, Md.). It has been tested to be free of viral or mycoplasma contamination, and was banked by Centocor's Cell Biology Services.

Complete IgG versions of the antibodies were engineered and cloned at Centocor: PHD126 stock solution was 1.7 mg/ml, PHD stock solution was 0.62 mg/mL, and cVaM was 10.09 mg/ML. All antibodies have been tested and have an LAL<4 EU/mg

PANC-1 cells were harvested at log phase by trypsinization, then washed one time in complete media and once in HBSS. PANC-1 cells (3.2×10⁷) cells were resuspended in 8.0 mL of ice-cold, sterile HBSS and mixed with 24 mL of ice-cold MATRIGEL (Becton Dickson) (final concentration was 1×10⁶ cells/mL). The final concentration of Matrigel was 10 mg/mL.

Mice were randomized into five groups (7 mice/group). Mice were anesthetized with Ketamine/xylazine (90/10 mg/kg, i.p.) and weighed. The mice were injected in each of two sites with 0.5 ml of Matrigel with tumor cell suspension (Groups 1 to 4). Group 5 animals were injected with Matrigel alone.

The injection sites were located on the dorsal side of the approximately 0.5 inches caudal to the last rib and 0.5 inches from the backbone on each side. Putting a finger over the injection site accelerates Matrigel polymerization and prevents any potential leakage. Due to the use of anesthesia and the injection of a cold substance, body temperature was maintained until the animal has regained consciousness. Under these conditions, the angiogenic factors are slowly released stimulating the process of angiogenesis and new vessel formation. Invasion of the gel plug by blood vessels occurs within 12-48 hours and neovascularization continues for up to 7-10 days following injection of the Matrigel.

Animals were injected with 0.2 cc (10 mg/kg) of each test article or 10 mL/kg of control on days 1 and 5 after tumor/Matrigel implant.

TABLE 6

| | | Ab Conc. | IP Dose | Treatment | Initiation |
|---|---|---|---|---|---|
| Group | Treatment | (mg/mL) | (mg/kg) | Frequency | Criteria |
| 1 | HBSS | n/a | 10 mL/kg | Days 1, 5 | Day 1 |
| 2 | PHD 126 | 1 | 10 | Days 1, 5 | Day 1 |
| 3 | PHD 127 | 1 | 10 | Days 1, 5 | Day 1 |
| 4 | cVaM | 1 | 10 | Days 1, 5 | Day 1 |
| 5 | HBSS | n/a | 10 mL/kg | Days 1, 5 | Day 1 |

All animals were weighed on Days 1, 5, and 9 (end of study). At termination the Matrigel plugs were excised and weighed. On day 9, all mice were euthanized by CO₂ asphyxiation. The animals were transported in a closed container (empty microisolator cage or sealed plastic bag) to the Oncology laboratory. While outside of the vivarium the animals were handled in a biosafety hood. The bodies were returned in a sealed plastic bag to the vivarium freezer when the work is completed. Plugs were surgically removed and weighed in a blinded fashion. Plugs were photographed and then processed for hemoglobin content and vessel length.

If at any time during the study an animal became moribund (>15% body weight loss, dyspnea, ataxia, tremors, etc) the PI would be notified and the animal euthanized. Disposition of the body was at the discretion of the PI.

Summary Following vessel density analysis we found that PHD 126 inhibited PANC-1 induced angiogenesis by approximately 60% (not statistically significant), while PHD 127 inhibited angiogenesis by approximately 88% relative to control antibody (p<0.05.)

PHD 127 inhibited angiogenesis by rates up to 88% (p<0.05, one-way ANOVA) demonstrating that an anti-Tissue Factor antibody has the ability to inhibit angiogenesis. Treatment with PHD 126 also inhibited angiogenesis in the same model by approximately 60% although the result was not statistically significant (FIG. 14).

These data demonstrate that proliferating cells produce a host response mediated by host TF leading to angiogenesis and, therefore, creating conditions permissive of tumor growth.

1. A method of treating a disease in a mammal characterized by an increase in vascularized tissue, which mammal is in need of such treatment, comprising administering to the mammal a tissue factor antagonist in an amount effective to inhibit the increase of said tissue in said mammal.

2. A method of claim 1, wherein the disease is selected from the group consisting of cancer, retinopathy, macular degeneration, rheumatoid arthritis and psoriasis.

3. A method for inhibiting the growth of a tumor in a mammal in need thereof comprising administering to the mammal a tissue factor antagonist in an amount effective to inhibit the growth of the tumor in said mammal.

4. The method of claim 3, wherein the tissue factor antagonist is a tissue factor monoclonal antibody or a fragment thereof.

5. The method according to claim 4, in which the antibody fragment is an Fab, Fab′, or F(ab′)2 fragment or derivative thereof.

6. The method according to claim 4, in which the antibody or fragment thereof prevents formation of the tissue factor: Factor VIIa: Factor X complex in plasma.

7. The method according to claim 4, in which the monoclonal antibody or fragment competes with monoclonal antibody TF8-5G9 for binding to human tissue factor.

8. The method according to claim 4, in which the monoclonal antibody is administered intravenously.

9. The method according to claim 4, in which the monoclonal antibody is administered in the amount of from 0.05 mg/kg to 12.0 mg/kg body weight.

10. The method according to claim 4, in which the monoclonal antibody is administered in a bolus dose followed by an infusion of said antibody.

11. The method according to claim 1 or 3, in which the mammal is a human patient.

12. The method of claim 3, wherein the tumor is a breast carcinoma or a pancreatic carcinoma.

13. The method of any of claims 1-12 wherein the antibody is administered in combination with a second anti-angiogenic agent.

14. A method of claim 13, where the second anti-angiogenic agent is a Mab capable of specifically binding the adhesion molecules containing alphaV.

15. A method of claim 13 where the second anti-angiogenic agent is a mAb capable of binding or functionally blocking other targets involved in cellular signaling pathways leading to angiogenesis.

16. The method of any of claims 3-12 wherein the antibody is administered in combination with antibody therapy, radiation therapy, a chemotherapeutic agent, a proteosome inhibitor, or a farnesyl transferase agent.

17. The use of a tissue factor antagonist in the manufacture of a medicament for the treatment of cancer, retinopathy, macular degeneration, arthritis or psoriasis, said medicament comprising an angiogenesis-inhibiting amount of said tissue factor antagonist.

18. The use of a tissue factor antagonist in the manufacture of a medicament for the treatment of tumors, said medicament comprising an amount of said tissue factor antagonist effective to inhibit tumor growth.
